(19) United States
(12) Patent Application Publication
(10) Pub. No.: US 2006/0047221 A1
(43) Pub. Date: Mar. 2, 2006

(54) New method to reduce complete blood count variation of peripheral blood sample

(75) Inventor: Yuandong Gu, Plymouth, MN (US)

Correspondence Address: HONEYWELL INTERNATIONAL INC., 101 COLUMBIA ROAD, P O BOX 2245, MORRISTOWN, NJ, 07962-2245, US

(73) Assignee: Honeywell International, Inc., Morristown, NJ (US)

(21) Appl. No.: 10/924,689

(22) Filed: Aug. 24, 2004

(51) Int. Cl. A61B 5/00

(52) U.S. Cl. 600/583

(57) ABSTRACT

The invention sets forth a method for reducing coagulation in a blood sample collected most conveniently from an acral body site such as the fingertip or heel, commonly referred to as capillary blood collection. The method includes the steps of applying an anticoagulant composition to the acral site, lancing the skin in contact with the anticoagulant composition and allowing blood and anticoagulant to mix on the skin at the site prior to collecting the blood for analysis.

### BACKGROUND OF THE INVENTION

The invention relates to blood sampling using technically simple procedures such as lancing the skin at a peripheral or “acral” body site. Such sampling methods are suitable for collecting samples in non-clinical environments by relatively untrained individuals. The ability to obtain blood samples in this manner facilitates economical and readily available point-of-care clinical laboratory health care testing. The present invention may be used in conjunction with state-of-the-art miniaturized hemo-analytical instruments intended for use in private homes, rural, or otherwise isolated settings where more invasive and risky methods and bulkier analytical instruments cannot or are unlikely to be used.

Advances in medical device miniaturization have made possible more accurate clinical blood analyses that use significantly smaller blood volumes than has been traditionally required. These advances have made possible the transfer of many clinical tests from large centralized labs to private doctor's offices, clinics and even patients' homes. This “point-of-care” health care philosophy seeks to maximize patient independence and control by maximizing the ease and economy of performing clinical tests for diagnosis and monitoring. It is desirable to provide additional point-of-care solutions and refine existing ones to benefit patients for whom visits to the hospital, clinic or testing lab are unduly difficult. Point-of-care solutions can significantly improve the economics of providing health care services in nonclinical settings. The invention herein described solves a significant practical issue that limits the clinical utility of small-volume blood samples obtained by lancing a patient's skin for blood analysis tests such as the Complete Blood Count (CBC).

### SUMMARY OF THE INVENTION

The invention described herein provides methods for reducing coagulation in a blood sample obtained by lancing the skin. The methods involve the initial application of an anticoagulant solution onto the skin site where blood is to be sampled, followed by lancing of the skin and blood collection. An anti-infective agent may be applied prior to application of the anticoagulant solution, after collection of the sample, or at both times. In other embodiments, the methods of the present invention are applied to blood sampling at peripheral or acral body sites such as the fingertips, heels, earlobes, or toes. In some embodiments the anticoagulant is ethylene diamine tetra-acetic acid (EDTA). The EDTA may be in the form of a salt in particular, a potassium, sodium, or lithium salt, or a mixture of these salts. In other embodiments, the anticoagulant is sodium citrate, acid citrate dextrose, citrate phosphate dextrose, low molecular weight heparin, heparin, ethyleneglycol-bis-(beta-aminoethylether)-N,N,N′,N′-tetra-acetic acid (EGTA), or 1,2-bis(o-aminophenoxy)ethane-N,N,N′,N′-tetra-acetic acid (BAPTA). In other embodiments the anticoagulant is a derivative or polymer of any of the aforementioned anticoagulants. A derivative of an anticoagulant is an anticoagulant that, while being chemically distinct from any of the aforementioned anticoagulants, has a chemical structure that has been based upon has been any of the aforementioned anticoagulants. A polymer of an anticoagulant is an anticoagulant that comprises any of the aforementioned anticoagulants covalently linked together. In some embodiments the anticoagulant solution further includes water, an anti-infective agent such as isopropyl alcohol, or mixtures thereof. To facilitate the clotting process after blood collection, the invention provides, in yet other embodiments, for application of a solution containing calcium ions or other non-toxic multivalent ions to the blood collection site.

### DETAILED DESCRIPTION OF THE INVENTION

The invention provides methods for reducing coagulation in blood samples obtained from acral body sites, thereby preserving the sample's compositional integrity and morphology. Blood is composed of a variety of living cells suspended in an aqueous medium that circulates through the heart, arteries and veins transporting nourishment, hormones, vitamins, antibodies, heat and oxygen to the body's tissues. Blood contains three main components—red blood cells, white blood cells, and platelets—suspended in a fluid called plasma. Red blood cells (RBCs) contain hemoglobin, a protein that carries oxygen to all the tissues of the body. White blood cells (WBCs) protect the body from invasion by foreign substances such as bacteria, fungi and viruses while mediating the immune response. There are five different types of white blood cells, each with its own function in protecting against infection: neutrophils (also known as “segs”—for segmented neutrophils, polymorphonuclear leukocytes (PMNs), or granulocytes), lymphocytes, monocytes, eosinophils, and basophils. Platelets are small irregularly-shaped cell-like bodies primarily responsible for maintaining the structural integrity of the circulatory system (hemostasis), through their central role in blood clotting. Platelets facilitate blood clotting by sticking to the lining of broken blood vessels (adhesion) and accumulating to form a barrier to bleeding (activation). Abnormalities in the number or morphology of any of these blood components can indicate the presence of potentially serious medical disorders.

A number of clinical laboratory procedures require the collection and analysis of a patient's blood. A complete blood count (CBC), for example, provides detailed information about the cellular components of blood. The CBC is one of the most commonly ordered clinical blood tests and provides important information about the most common blood components.

The CBC is used as a broad screening test to check for such disorders as anemia (decrease in red blood cells or hemoglobin), infection, leukemia, and many other diseases. It is actually a panel of tests that examine different parts of the blood. The CBC encompasses all or some of the following measurements: (1) a white blood cell (WBC) count expressed as number (thousands) per microliter of blood; (2) a white blood cell differential which classifies the WBCs into each type (neutrophils, lymphocytes, etc.) expressed as percentages of all WBCs; (3) a red blood cell (RBC) count expressed as millions per microliter; (4) a hemoglobin value expressed in grams per deciliter of blood; (4) hematocrit (HCT) or percentage of blood volume comprising red blood cells; (5) a platelet count expressed as thousands per microliter; (6) mean corpuscular volume (MCV)—the volume amount of hemoglobin inside the red blood cells expressed in femtoliters (10⁻¹⁵ liters); (7) mean corpuscular hemoglobin concentration (MCHC) or the percentage of all hemoglobin that is carried within the red blood cells; and (8) the red cell distribution width (RDW) which quantifies the variation in RBC size (RDW=100×standard deviation of RBC size/mean RBC size).

In adults, blood tests such as the CBC require the collection of a representative sample of the patient's blood—obtained most often by venipuncture. In the venipuncture procedure, blood is drawn from a vein, usually from the inside of the elbow (antecubital) or the back of the hand. The puncture site is cleaned with antiseptic, and a tourniquet (usually an elastic band) or blood pressure cuff is placed around the upper arm to apply pressure and restrict blood flow through the vein which causes veins below the tourniquet to distend (fill with blood). Next, a needle is inserted into the vein, and the blood is collected in an air-tight vial or a syringe usually containing an additive specific to the type of blood analysis to be performed. While the needle is inserted, the tourniquet is removed to restore circulation. Once the blood has been collected, the needle is removed, and the puncture site is covered to stop any bleeding. For infants and young children, venipuncture is not recommended and instead an acral body skin site such as a fingertip or heel is cleansed with antiseptic and punctured with a sharp needle or blade—collectively referred to as a lancet. The blood is collected in a pipette (small tube), on a slide, onto a test strip, or into a small container all of which may contain an additive such as an anticoagulant depending on the subsequent analysis to be performed. After collection, bandage may be applied to the puncture site to avoid continued bleeding.

Venipuncture is associated with slight risks for excessive bleeding, hematoma, fainting, light-headedness, and infection. A higher likelihood exists for certain individuals with hard-to-locate antecubital veins to sustain multiple punctures. Under all circumstances, it is strongly recommended that venipuncture be performed in a controlled clinical setting by a skilled and experienced health care provider or technician. The acral lancing or fingerprick procedure is simpler, less invasive, has fewer risks, and requires only minimal skill to implement. Thus, it can be performed far more economically and in non-clinical field settings as compared to venipuncture. The traditional fingerprick procedure, however, initiates coagulation in the blood sample that will adversely effect many blood measurements including the CBC. This is because blood coagulation entraps plasma components like RBCs, WBCs, and platelets in a rapidly forming protein (fibrin) matrix. Entrapment compromises blood sample integrity, resulting in inaccurate and distorted blood analysis measurements. The venipuncture procedure minimizes coagulation by collecting blood with an anticoagulant-treated needle directly into a vial containing an anticoagulant. Furthermore, the relatively large volume of blood collected during venipuncture tends to mask the effects of incidental coagulation initiated by vessal wall rupture, and needle and vial inside-wall surface contact.

Collecting blood samples from an acral body site such as the finger or heel usually results in the sampled blood contacting the patient's outer skin surface before collection. This blood/skin contact, together with Tissue Factor released from damaged vessel walls and skin, initiates the cascade of biochemical reactions that result in blood clotting. A significant fraction of the relatively small volume (typically 50 microliters) of blood collected in this manner will undergo platelet adhesion, fibrin formation, and blood component entrapment. Such blood samples will yield CBC results significantly distorted by these extra-corporeal processes. CBC results that do not accurately reflect the patient's in vivo hematological state will adversely affect the health-care professional's ability to formulate a reliable diagnosis.

Accordingly, it would be desirable to provide a quick and simple method for sampling blood that benefits from acral body site sampling while minimizing collateral coagulation resulting from blood-skin contact. Such a method would improve the reliability of a variety of point-of-care blood testing procedures.

The methods of the present invention utilize a liquid composition containing an anticoagulant designed to reduce coagulation of blood at the skin surface site where the blood is to be collected.

After application of the liquid composition, the skin is lanced in the usual manner. In certain embodiments of the present invention, the composition can be allowed to dry before lancing. After lancing, blood is allowed to collect at the skin site where it contacts the anticoagulant present either in liquid form or dried on the skin surface. The present invention allows the blood sample to contact the skin prior to collection while avoiding the initiation of significant coagulation within the sample. This is because anticoagulant is present within the blood sample from the moment it reaches the skin surface. Normally, blood contact with tissue at the wound site and on the skin initiates the cascade of biochemical events resulting in fibrin formation and coagulation. In contrast, the blood sample obtained using the method of the present invention is immediately protected from coagulation that would otherwise result from collateral contact with surfaces such as skin, the collection device (e.g. pipette, needle, container), or the analytical device (e.g. hemocytometer). This provides enhanced protection from fibrin formation and blood component entrapment over that otherwise available when simply collecting the blood into a vial, pipette or other container (Vacutainer®) containing anticoagulant.

The term anticoagulant as used herein refers to compounds capable of inhibiting one or more of the steps involved in blood clotting. An anticoagulant is a substance that prevents blood from coagulating or “clotting.” There are two major methods commonly used to prevent coagulation (i) chelating (binding) or precipitating calcium and making it unavailable for the coagulation process or (ii) inhibiting formation of the thrombin needed to convert fibrinogen to fibrin. The anticoagulant can be dissolved or suspended in a liquid composition. The anticoagulant can be a man-made or naturally occurring compound. Examples of known anticoagulants include ethyleneglycol-bis-(beta-aminoethylether)-N,N,N′,N′-tetra-acetic acid (EGTA), 1,2-bis(o-aminophenoxy)ethane-N,N,N′,N′-tetra-acetic acid (BAPTA) heparin, lithium heparin, sodium heparin, sodium citrate, acid citrate dextrose solutions (ACD) and variants, citrate phosphate dextrose solutions and variants, ethylene diamine tetra-acetic acid (EDTA) and variants including its potassium, sodium, and lithium salts. Potassium oxalate and ammonium oxalate, two anticoagulants used in clinical settings, are severe skin irritants and are less likely to be used in the methods of the present invention.

In one embodiment of the invention, the anticoagulant used is acid citrate dextrose (ACD). ACD is a solution of citric acid, sodium citrate and dextrose in water. It is used as an anticoagulant for the preservation of stored whole blood and for extracorporeal procedures to selectively remove platelets from whole blood (“plateletpheresis”). A typical ACD formulation contains 0.008 grams per milliter (g/ml) citric acid, 0.023 g/ml sodium citrate, and 0.022 g/ml dextrose. Another embodiment of the present invention uses a citrate phosphate dextrose solution (CPD) as an anticoagulant. A typical CPD formulation contains 0.003 g/ml citric acid, 0.026 g/ml sodium citrate, 0.002 g/ml sodium biphosphate, and 0.032 g/ml dextrose. Adenine is often added (typical concentration of 0.03 g/100 ml) to both ACD and CPD solutions as a red blood cell preservative. Modified ACD and CPD solutions are used for long term preservation of whole blood or red cells and sold commercially as Adsol®, Nutricel®, and Optisol® among others.

In yet another embodiment of the present invention, a solution of sodium citrate and water is used as the anticoagulant. Anticoagulant solutions containing sodium citrate (including ACD and CPD solutions and variants) prevent coagulation by binding calcium. They require precise mixing with whole blood in order to be effective. Because of the relatively high sodium citrate concentrations necessary to achieve an effective anticoagulant effect, ACD, CPD and sodium citrate solutions should be iso-osmolar relative to the RBCs, otherwise RBC shrinkage will result and be reflected in CBC measurements as artifactually low hematocrit values. A 0.109 mM solution of sodium citrate in water is iso-osmolar with red blood cells. When using appropriate sodium citrate solutions, care should be taken that the solvent is not allowed to evaporate significantly. Even partial evaporation after application of an iso-osmolar sodium citrate solution will increase the osmolarity of the liquid composition remaining with deleterious effects on subsequently measured hematocrit values.

A preferred embodiment of the present invention uses low-molecular-weight (LMW) heparin as the anticoagulant. Heparin is a sulfated glycosaminoglycan of mixed polysaccharide nature varying in molecular weights and composed of polymers of alternating derivatives of D-glycosamine and L-iduronic acid or D-glucuronic acid. It is released by mast cells and basophils in the blood and is present in many tissues, especially the liver and lungs. Heparin is a mixture of several active agents, some of which have potent anticoagulant properties that result from binding to and greatly enhancing the activity of antithrombin III and from inhibition of a number of coagulation factors, particularly activated factor X (factor Xa). Low-molecular-weight heparin is preferred over normal heparin as the latter tends to cause white blood cell clumping leading to distortion of white blood cell counts. Low-molecular-weight heparin is derived from standard heparin through either chemical or enzymatic depolymerization. Whereas standard heparin has a molecular weight of 5,000 to 30,000 daltons, LMW-heparin ranges from 1,000 to 10,000 daltons, resulting in chemical and physical properties distinct from those of traditional heparin. LMW heparin binds less strongly to protein, and interacts less with platelets. Degradation and neutralization of anticoagulant activity is less of a problem when LMW heparin is used over standard heparin. LMW heparin, like standard heparin, binds to antithrombin III. However, LMW heparin inhibits thrombin to a lesser degree (and Factor Xa to a greater degree) than standard heparin. Sodium and calcium heparin sodium exist as white or pale-colored, amorphous, hygroscopic powders having a faint odor. Both are soluble in water and practically insoluble in alcohol. Heparin potency is expressed in terms of USP Heparin units and values are obtained by comparing against a standard USP reference. A typical blood-heparin mixture for anticoagulant purposes during blood collection is 70-150 USP units per 10- to 20 ml sample.

A particularly preferred embodiment of the invention uses the anticoagulant ethylene diamine tetra-acetic acid (EDTA). EDTA is a metal-complexing agent which inhibits blood coagulation by chelating calcium ions to form soluble complexes. Because free calcium is necessary for the formation of fibrin, clotting cannot take place in the presence of sufficient quantities of EDTA. The EDTA is preferably present in solution as a salt or as a mixture of salts. Preferred salts of EDTA include but are not limited to Sodium (Na), Potassium (K) or Lithium (Li) salts of EDTA. Dipotassium EDTA is particularly preferred in the practice of the present invention. Dipotassium EDTA and tripotassium EDTA are readily soluble in water and alcohol.

As an example of formulating an EDTA solution appropriate for the methods of the present invention, it is noted that an EDTA concentration of about 1.5 milligrams (mg) EDTA per milliliter (ml) of blood is effective in preventing coagulation. A typical fingerprick blood sample of 100 microliters (μl) will therefore require about 0.15 milligrams of EDTA to prevent coagulation. Assuming that the median thickness of the EDTA solution present on the skin after application is 0.01 centimeters (cm) and that it covers an area of 0.8 cm², then the total volume of anticoagulant solution applied would be approximately 0.008 cm³. Since at least about 0.15 mg of EDTA is required in this volume, the concentration of EDTA required in the liquid composition applied to the skin before lancing will be about 18.75 mg/cm³ (0.15 mg/0.008 cm³) or 1.9% by weight (w/v) EDTA. Alternatively a solution of EDTA in a common 70% isopropyl alcohol anti-infective solution can be used. Using 70% isopropyl alcohol, only one-third of the liquid composition applied to the skin will be water—about 0.0026 cm³. To prepare such a solution, a solution of EDTA in water is made having a concentration of 58 mg/cm³ (0.15 mg EDTA/0.0026 cm³ water) or 5.6% w/v EDTA. Thirty millilters of this aqueous solution is mixed with 70 ml of 100% isopropyl alcohol to give a 1.9% w/v EDTA, 70% isopropyl alcohol solution suitable for use in the method of the present invention.

The liquid composition applied to the skin site before lancing can contain an anticoagulant and an anti-infective agent. In preferred embodiments, a solution of 0.1 to 30% w/v of dipotassium EDTA (K₂EDTA) in 70% isopropyl alcohol is prepared. In more preferred embodiments, a solution of about 1 to about 10% w/v of K₂EDTA in 70% isopropyl alcohol is used. In the most preferred embodiments a solution of about 1 to about 3% K₂EDTA in 70% isopropyl alcohol is used.

A liquid composition of isopropyl alcohol, water and K₂EDTA may evaporate when applied to a warm skin surface resulting in K₂EDTA precipitate remaining on the skin. When the skin is subsequently lanced, the accumulating blood will contact the EDTA salt, which will dissolve back into solution. Using the method of the present invention, EDTA is immediately present in the blood as it collects on the skin surface. Osmolarity considerations are generally not a problem when using EDTA salt solutions since the concentration necessary for effective anticoagulation is so small.

Capillary blood collection utilizes an acral body site as a sampling locus. The word “acral” refers to the extremities (e.g. hands, feet, ears, nose). Acral blood collection is often termed capillary blood collection as lancing of acral body skin sites will sample capillary blood. In a preferred embodiment of the present invention, the 3^{rd} and 4^{th} fingers of the non-dominant hand, counting from the thumb, are used for blood collection in adults (The 2^{nd} (index) finger tends to have thicker, callused skin and the 5^{th} finger tends to have less soft tissue overlying the bone) and the heel is used in infants and toddlers. To perform the procedure, the patient should be in a sitting position or lying down with arms hyper-extended. Neither the tip of the finger nor the center of the finger should be used as a lancing site. Regions of the finger with minimal soft tissue, where vessels and nerves are located, and where the bone is closer to the surface should be avoided as well. Fingers that are cold or cyanotic (oxygen depleted, bluish in color), swollen, scarred, or covered with a rash should not be used. The ideal lancing site is just off the center of the finger pad.

Sterile lancets used for acral site sampling are available in 21 to 26 gauge corresponding to 0.81 to 0.46 millimeters (mm) outside diameter (O.D.) respectively. Typical penetration depths range from 1.2 to 2.8 mm. Optimal needle size is made based on the patient's age, the intended blood-sampling skin site, and the physical quality of the patient's skin, (i.e. delicate, rough, calloused). Most commercially available lancets are contained in some type of housing that protects against accidental pricks, contamination and inadvertent reuse. Using the lancet, the puncture should be made perpendicular to the ridges of the fingerprint so that the drop of blood does not run down the ridges of the fingertip. Although some massaging of the finger is permitted to encourage blood to accumulate, excessive pressure that may squeeze tissue fluid into the drop of blood should be avoided. When a sufficient amount of blood has accumulated, usually about 50 μl, it is collected either by inverting the finger to allow the accumulated blood to drop into a sterilized vial which, for CBC purposes contains K₂EDTA. Alternatively, a sterilized glass capillary tube with anticoagulant coating its inside surface, is placed adjacent to the accumulated blood, thereby allowing the blood to move into the tube by capillary action. The container is then capped, rotated and inverted to mix the collected blood sufficiently with any additional anticoagulant or other additive contained within.

A further embodiment of the invention comprises preceding the application of anticoagulant to the skin surface with the application of an anti-infective agent. Such an agent can comprise one or both of an antiseptic and a disinfectant. Antiseptics and disinfectants are used to reduce the risk of infection. Antiseptic refers to an agent used to clean living tissue while disinfectant refers to an agent used to clean a surface other than living tissue. Examples of common disinfectants include sodium hypochlorite (chlorine bleach) and hydrogen peroxide. To prevent contamination by common skin bacteria, antiseptics are used to clean the patient's skin before puncture. The most commonly used antiseptic is 70% isopropyl alcohol. Isopropyl alcohol is a bacteriostatic since it inhibits growth of bacteria but does not kill them. Prepackaged alcohol “prep pads” are commonly used in clinical settings for this purpose.

Stronger antiseptics are used when more stringent infection control is needed, such as for blood cultures or arterial punctures. Betadine (povidone-iodine solution) is commonly used for these cases. For patients who are allergic to iodine, chlorohexidine gluconate or benzalkonium chloride (Zephiran®) is available. These antiseptics are harsher to the skin so they should be washed off with isopropyl alcohol after collection.

Optionally, an anti-infective agent can be applied before application of the anticoagulant liquid composition. A preliminary, anti-infective application step may be necessary if the anticoagulant possesses poor stability in the presence of common antiseptic agents such as isopropyl alcohol. In preferred embodiments of the present invention, the anti-infective agent is in liquid form and is allowed to dry before application of the anticoagulant composition since this aids in disinfecting the skin.

In certain embodiments, the methods of the present invention can include the optional step of applying a cleansing solution that contains calcium ions or other non-toxic multivalent ions such as iron, magnesium, and aluminum ions. For example, a 70% isopropyl alcohol solution containing calcium ions can be applied to the blood collection site as a last step. In preferred embodiments a solution of 0.1 to 1% w/v CaCl₂ is used for this step. The presence of calcium ions is intended to replace calcium ions functionally lost as a result of the action of an anticoagulant such as EDTA that chelate or otherwise make calcium unavailable for the blood coagulation cascade. Replacing calcium ions enables coagulation to proceed, thereby allowing a clot to form more rapidly than if no calcium ions are supplied. Different salts of the calcium ions having good solubility in solutions of isopropyl alcohol may be used. These include but are not limited to chlorides, nitrates, gluconates, and acetates. Since only a trace amount of these ions is needed to counteract the chelating effect of certain anticoagulants (e.g. EDTA), the solubility of the above salts in commonly used isopropyl alcohol solutions will be sufficient. Other solutions containing these ions may be prepared using methods known in the art.

Alternatively, calcium ion salts may be incorporated in a bandage applied immediately after blood collection or alternatively, after application of an anti-infective agent. A bandage could provide a localized, high multivalent ion concentration to effectively counteract certain anticoagulants applied to the site before blood collection. Blood flowing through the puncture will dissolve multivalent ions in the bandage, facilitating the coagulation process. In yet another embodiment, a “wipe” or towelette containing multivalent ions in solution may be used. Upon wiping the surface of the sampled skin site, the multivalent ions will facilitate coagulation. Bleeding is also minimized by applying pressure to the puncture site. This can be done using a gauze pad folded into quarters. When the bleeding stops, gauze is taped over the puncture site with paper tape or an adhesive bandage.

Although illustrative embodiments of the present invention have been described herein, it is to be understood that the invention is not limited to those precise embodiments, and that various other changes and modifications may be effected therein by one skilled in the art without departing from the scope or spirit of the invention.

### EXAMPLES

### Example 1

### Method for Minimizing Coagulation in a Blood Sample Obtained from an Adult's Finger Utilizing a Liquid Composition Containing 70% Isopropyl Alcohol and 2% K₂EDTA

The 2% K₂EDTA liquid composition is prepared using routine methods. The patient sits in a chair and is asked to hyperextend her non-dominant hand. The middle finger is selected as the skin surface site to be lanced. The patient is directed to wash her hands with soap and warm water. Still cold to the touch, the patients hand is wrapped in a warm, moist towel (not more than 40° C./105° F.) for two minutes while holding her hand in a downward position to allow gravity to increase blood supply to the hand. The technician performing the collection puts on latex gloves. The patient's finger is wiped clean using a cotton swab soaked in a solution of 70% isopropyl alcohol. The finger is allowed to air-dry. A 2% K₂EDTA anticoagulant liquid solution is then wiped onto the pad of the middle finger using a cotton swab. A 21 guage (0.81 mm O.D.) lancet is used to puncture the finger pad at least 2.5 mm left or right of an imaginary line positioned on the center of the finger and running parallel to its length. The lancet is chosen and manipulated to puncture the skin to a depth of approximately 1.8 mm. Blood is allowed to accumulate on the skin surface. When a sufficient amount of blood, about 50 to 100 μl, has accumulated, the finger is inverted and the blood allowed to drop into a sterilized Eppendorf® vial containing no anticoagulant. The vial is sealed and labeled for further analysis. The puncture site is then wiped clean with a 70% isopropyl alcohol solution. A dry sterile gauze pad is taped firmly onto the puncture site.

### Example 2

### Method for Minimizing Coagulation in a Blood Sample Obtained from an Infant's Heel Utilizing a Liquid Composition Containing 70% Isopropyl Alcohol and 2% K₂EDTA

A blood-sampling site is selected from regions comprising at least 1 cm on either side of an imaginary line placed on the bottom of an infant's foot and along its length. The area is cleaned thoroughly with soap and warm water and dried with a clean towel. A cotton swab is used to apply a liquid composition of 2% K₂EDTA and 70% isopropyl alcohol to the sample site. Using a 0.25 gauge (0.51 mm O.D.) lancet the skin is punctured to a depth of approximately 1.0 mm. Blood is allowed to accumulate at the puncture site. Blood is collected using a capillary tube attached to a container. The puncture site is wiped using a 70% isopropyl alcohol solution containing 1% w/v CaCl₂.

We claim:

1. A method for reducing coagulation in a blood sample obtained by lancing a skin surface site of an animal comprising:
(a) applying a liquid composition comprising an anticoagulant to said skin surface site;
(b) lancing the skin surface at said site such that said blood contacts said anticoagulant on said skin surface;
(c) collecting said blood sample.

2. The method of claim 1 further comprising the step of applying an anti-infective agent to said skin site before application of said liquid composition.

3. The method of claim 1 wherein said skin site is an acral site.

4. The method of claim 3 wherein said acral site is selected from the group consisting of said animal's fingertips, heels, earlobes, or toes.

5. The method of claim 1 wherein said anticoagulant comprises ethylene diamine tetra-acetic acid (EDTA).

6. The method of claim 5 wherein said EDTA is in the form of a salt in said liquid composition.

7. The method of claim 6 wherein said salt comprises the potassium salt of EDTA.

8. The method of claim 6 wherein said salt comprises the sodium salt of EDTA.

9. The method of claim 6 wherein said salt comprises the lithium salt of EDTA.

10. The method of claim 6 wherein said salt comprises a mixture of two or more salts of EDTA.

11. The method of claim 1 wherein said anticoagulant is selected from the group consisting of EDTA, sodium citrate, acid citrate dextrose, citrate phosphate dextrose, low molecular weight heparin, heparin, ethyleneglycol-bis-(beta-aminoethylether)-N,N,N′,N′-tetra-acetic acid (EGTA), or 1,2-bis(o-aminophenoxy)ethane-N,N,N′,N′-tetra-acetic acid (BAPTA).

12. The method of claim 1 wherein said liquid composition further comprises isopropyl alcohol.

13. The method of claim 1 wherein said liquid composition further comprises water.

14. The method of claim 1 further comprising the step of applying an anti-infective agent to said skin site following collection of said blood sample.

15. The method of claim 14 wherein said anti-infective agent comprises an isopropyl alcohol solution.

16. The method of claim 14 wherein said anti-infective agent further comprises calcium ions.

17. The method of claim 14 wherein said anti-infective agent further comprises non-toxic multivalent ions.

18. A method for reducing coagulation in a blood sample obtained by lancing a fingertip comprising the steps of:
(a) applying a liquid composition comprising an anticoagulant to said fingertip;
(b) lancing the skin at said fingertip such that said blood contacts said anticoagulant on said skin surface;
(c) collecting said blood sample.

19. The method of claim 18 further comprising the step of applying an anti-infective agent to said skin site before application of said liquid composition.

20. The method of claim 18 wherein said anticoagulant comprises EDTA.

21. The method of claim 20 wherein said EDTA is in the form of a salt in said liquid composition.

22. The method of claim 21 wherein said salt comprises the potassium salt of EDTA.

23. The method of claim 21 wherein said salt comprises the sodium salt of EDTA.

24. The method of claim 21 wherein said salt comprises the lithium salt of EDTA.

25. The method of claim 21 wherein said salt comprises a mixture of two or more salts of EDTA.

26. The method of claim 18 wherein said anticoagulant is selected from the group consisting of EDTA, sodium citrate, acid citrate dextrose, citrate phosphate dextrose, low molecular weight heparin, heparin, ethyleneglycol-bis-(beta-aminoethylether)-N,N,N′,N′-tetra-acetic acid (EGTA), or 1,2-bis(o-aminophenoxy)ethane-N,N,N′,N′-tetra-acetic acid (BAPTA).

27. The method of claim 18 wherein said liquid composition further comprises isopropyl alcohol.

28. The method of claim 18 wherein said liquid composition further comprises water.

29. The method of claim 18 further comprising the step of applying an anti-infective agent to said skin site following collection of said blood sample.

30. The method of claim 29 wherein said anti-infective agent comprises an isopropyl alcohol solution.

31. The method of claim 29 wherein said anti-infective agent further comprises calcium ions.

32. The method of claim 29 wherein said anti-infective agent further comprises any non-toxic multivalent ions.

33. A method for reducing coagulation in a blood sample obtained by lancing a fingertip comprising the steps of:
(a) applying a liquid composition comprising ethylene diamine tetra-acetic acid (EDTA) to said fingertip;
(b) lancing the skin at said fingertip such that said blood contacts said EDTA on said skin surface;
(c) collecting said blood sample.

34. The method of claim 33 further comprising the step of applying an anti-infective agent to said skin site before application of said liquid composition.

35. The method of claim 33 wherein said liquid composition further comprises isopropyl alcohol.

36. The method of claim 33 wherein said liquid composition further comprises water.

37. The method of claim 33 further comprising the step of applying an anti-infective liquid agent to said skin site following collection of said blood sample.

38. The method of claim 37 wherein said liquid anti-infective agent comprises an isopropyl alcohol solution.

39. The method of claim 37 wherein said anti-infective agent further comprises calcium ions.

40. The method of claim 37 wherein said anti-infective agent further comprises non-toxic multivalent ions.
